(19) United States
(12) Patent Application Publication
(10) Pub. No.: US 2025/0201398 A1
(43) Pub. Date: Jun. 19, 2025

(54) Addressing Complications of Multiple Healthcare Action Items

(71) Applicant: GoForward, Inc., San Francisco, CA (US)

(72) Inventors: Adrian Aoun, San Francisco, CA (US); Chandrashekar Raghavan, Alameda, CA (US); Sudarshan Narasimha Raghavan, Dublin, CA (US)

(21) Appl. No.: 18/935,408

(22) Filed: Nov. 1, 2024

Related U.S. Application Data
(60) Provisional application No. 63/609,468, filed on Dec. 13, 2023.

(51) Int. Cl. G16H 40/63; G16H 10/60; G16H 70/20

(52) U.S. Cl. CPC G16H 40/63; G16H 10/60; G16H 70/20

(57) ABSTRACT

Embodiments relate to addressing complications associated with performing multiple healthcare action items for a patient or a visitor of a medical station. Applications executed on the medical station generate initial healthcare action items using patient and diagnostic information. Complications arising from the initial healthcare action items are identified, and the initial healthcare action items are converted into revised healthcare action items that reduces the complications. The revised healthcare action items may reduce adverse outcomes compared to the initial healthcare action items, thereby improving the healthcare service provided by the medical station.

### CROSS-REFERENCE TO RELATED APPLICATION

This application claims priority under 35 U.S.C. § 119(e) to U.S. Patent Application No. 63/609,468, filed on Dec. 13, 2023, which is incorporated by reference herein in its entirety.

### BACKGROUND

Embodiments relate to managing healthcare action items, and more specifically to managing healthcare applications deployed in modular medical stations.

A healthcare protocol refers to a standardized set of guidelines, rules, or procedures that outline recommended steps and actions to be followed in diagnosing, treating, managing, or preventing various medical conditions or situations. Such healthcare protocols are generally used to ensure a consistent level of care across different healthcare settings and different healthcare professionals. The healthcare protocols may apply to various areas of healthcare including clinical diagnosis and treatment, addressing of emergency medical situations, care process for certain medical conditions, and prevention or management of chronic conditions such as diabetes or hypertension. Applying a healthcare protocol to the health data of a person may result in certain healthcare action items or protocol actions such as prescribing medications, recommending reduction or increase of certain exercises, conducting further examination for more accurate diagnosis, and performing of procedures like surgeries.

Multiple healthcare action items or protocol activities based on individual healthcare protocols may have conflicts, be redundant or overly complicate the compliance. Such issues in the action items or protocol activities may not be easily identifiable to healthcare professionals, resulting in inadequate or unnecessary waste of resources and impeding the treatment of patients or even harming the patients.

### SUMMARY

Embodiments relate to managing healthcare action items by addressing complications associated with multiple healthcare action items. A plurality of initial healthcare action items are generated for a patient or a visitor of a medical station by a plurality of healthcare applications using patient information and diagnostic information of the patient or the visitor. The complications caused by performing the initial healthcare action items on the patient or the visitor are determined. In response to detecting the complications, the initial healthcare action items are converted to one or more revised healthcare action items where performing of the revised healthcare action items reduces or eliminates the complications relative to the performance of the initial healthcare action items.

In one or more embodiments, each of the initial healthcare applications covers different fields of healthcare operations performed by the medical station.

In one or more embodiments, at least a subset of the initial healthcare action items is received from a protocol derived from one or more of medical professionals, medical service providers, medical publications or public entities.

In one or more embodiments, the complications caused by the performance of the plurality of initial healthcare action include at least one of: conflicts between the initial healthcare action items, redundancy in the plurality of initial healthcare action items, and an increase in complexity associated with performing the initial healthcare action items together.

In one or more embodiments, the converting of the initial healthcare action items into the revised healthcare action items includes at least one of: removing a subset of the plurality of initial healthcare action items according to priority, merging at least a subset of the initial healthcare action items, modifying a sequence of performing the initial healthcare action items, or replacing one or more of the initial healthcare action items.

In one or more embodiments, interactions or interchangeability between healthcare action items are stored in a database. The converting of the plurality of the initial healthcare action items is performed based on the stored interactions or the interchangeability.

In one or more embodiments, the converting of the plurality of the initial healthcare action items includes applying rules to the determined complications to generate the one or more revised healthcare action items.

In one or more embodiments, the patient information is updated according to the one or more revised healthcare action items.

In one or more embodiments, management operations of the medical station are performed. Analysis is also performed to determine the efficacy of treatments according to the initial healthcare action items.

### BRIEF DESCRIPTION OF THE DRAWINGS

FIG. 1 is a block diagram illustrating the overall network environment including medical stations, according to one embodiment.

FIG. 2 is a block diagram illustrating components of a backend server, according to one embodiment.

FIG. 3 is a block diagram illustrating components of a medical station, according to one embodiment.

FIG. 4 is a conceptual diagram of managing healthcare action items, according to one embodiment.

FIG. 5 is a flowchart illustrating operations of managing healthcare action items, according to one embodiment.

### DETAILED DESCRIPTION OF EMBODIMENTS

Embodiments are described herein with reference to the accompanying drawings. Principles disclosed herein may, however, be embodied in many different forms and should not be construed as being limited to the embodiments set forth herein. In the description, details of well-known features and techniques may be omitted to avoid unnecessarily obscuring the features of the embodiments. In the drawings, like reference numerals in the drawings denote like elements. The shape, size and regions, and the like, of the drawing may be exaggerated for clarity.

Embodiments relate to addressing complications associated with performing multiple healthcare action items for a patient or a visitor of a medical station. Applications executed on the medical station generate initial healthcare action items using patient and diagnostic information. Complications arising from the initial healthcare action items are identified, and the initial healthcare action items are converted into revised healthcare action items that reduces or eliminates the complications. The revised healthcare action items may reduce adverse outcomes compared to the initial healthcare action items, thereby improving the healthcare service provided by the medical station.

A healthcare action item refers to an action item or an activity item to manage, treat, improve or diagnose the health conditions of a person. The healthcare action item may include, among others, prescription of medication, assigning of physical/mental exercises, assigning of nutrition plans, referring to a health specialist, recommending procedures (e.g., surgery or follow-up examinations), extraction and testing of bodily samples (including blood, stool or urine), imaging (e.g., X-Ray, CT or MRI), vaccination, invasive or non-invasive surgery, and the change of medication including dosage, frequency and form.

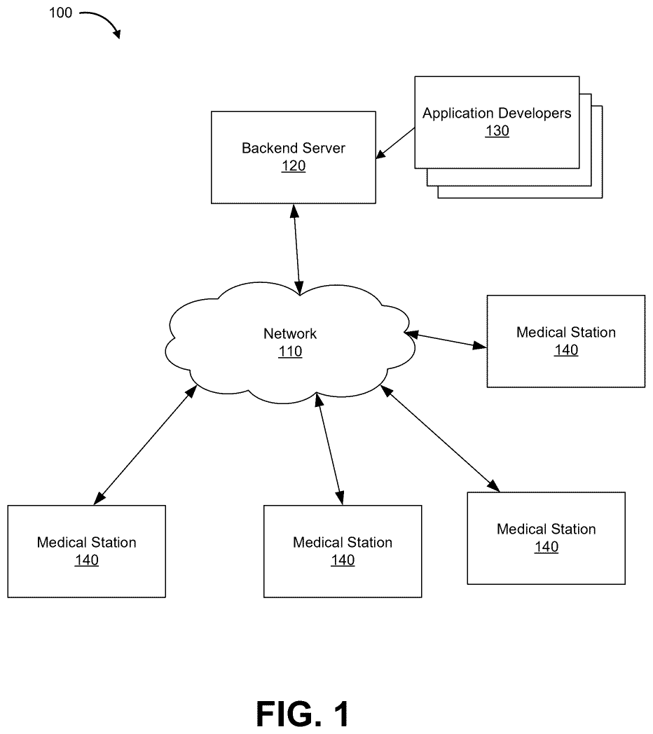

FIG. 1 is a block diagram illustrating the overall network environment 100 including medical stations 140, according to one embodiment. Network environment may include, among other components, a backend server 120, application developer devices 130 and medical stations 140. Some or all of these components communicate over network 110. Although application developer devices 130 are illustrated in FIG. 1 as communicating with backend server 120 directly, application developer devices 130 may communicate with backend server 120 via network 110. The network environment may include other components not illustrated in FIG. 1, such as databases in different medical facilities that store diagnostic information and/or patient information.

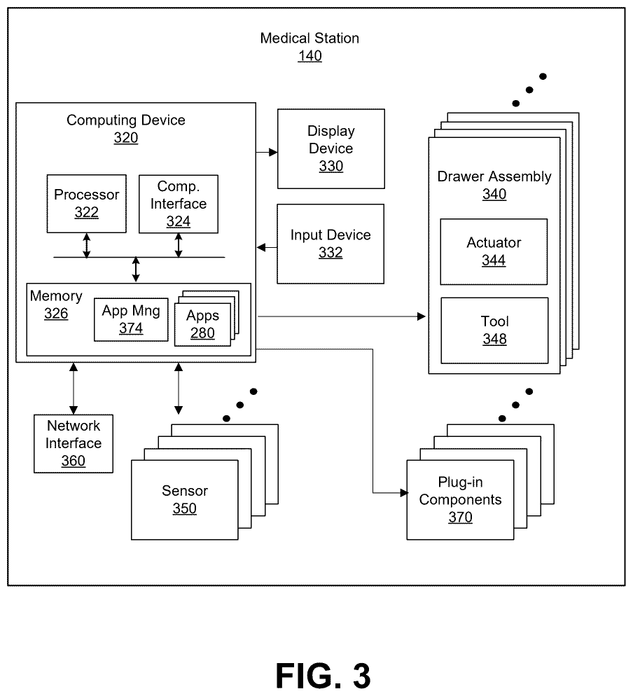

A medical station described herein is a prefabricated modular structure equipped with sensors and medical tools to provide medical services to patients or visitors. Medical station 140 may have a predetermined dimension, such as 10 feet by 15 feet or any other suitable dimensions, that can be installed inside or outside a building. An example structure of medical station 140 is described below in detail with reference to FIG. 3. Medical station 140 communicates with backend server 120 to send and receive login information of the patient or visitor accessing medical station 140, send and receive information associated with applications launched at medical station 140, send information on inventories of medical tools in medical station 140, and receive applications 280 for execution.

Due to their relatively small size and ease of installation, medical stations 140 may be beneficially installed at diverse geographical locations at relatively low cost. Medical stations 140 may be mass-produced at a factory and have forms and structures adapted for deployment with reduced construction fees. A patient or a visitor may visit one of many sites installed with medical stations to receive medical services. In one or more embodiments, medical stations 140 may be operable shortly after plugging them into power outlets and setting up wireless or wired communication. Furthermore, medical stations 140 can be relocated to another site with ease, depending on, for example, changing needs at different sites. In some embodiments, the medical stations 140 may be deployed outdoors, may be mobile or otherwise movable (e.g., via built-in wheels or other transportation infrastructure), may be self-leveling (e.g., via one or more position or orientation adjustment mechanisms), and/or may be pre-assembled or pre-manufactured for on-site assembly.

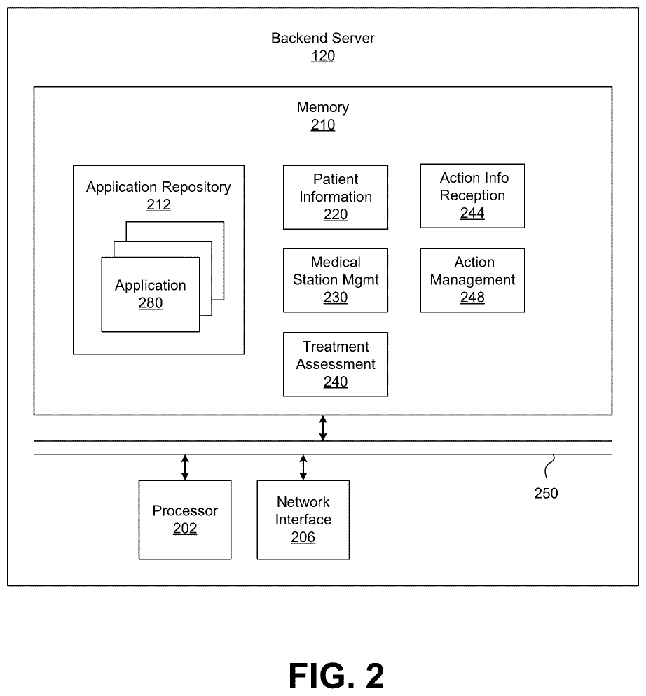

Backend server 120 is a computing device that facilitates or supports medical stations 140 to provide medical services to its patients or visitors. Backend server 120 may store various applications 280 from various sources, including application developer devices 130, and may send the applications 280 to medical stations 140 for deployment. Backend server 120 may also evaluate the performance of various applications 280 by collecting information received from medical stations 140, and update/discard applications 280 upon performance and/or demand. Backend server 120 may also perform management of healthcare action items generated by applications 280 deployed in medical stations 140. The components and functions of backend server 120 are described below in detail with reference to FIG. 2.

Application developer devices 130 are computing devices used by various entities such as healthcare service providers to construct applications 280 and provide them to backend server 120. Each of application developer devices 130 may be associated with developing and providing healthcare applications of different types and/or cover different fields of healthcare operations that may be performed with medical station 140. For example, an application developer device may construct and provide a healthcare application dedicated to cardiovascular health while another or the same application developer device may construct and provide another healthcare application related to diagnosis and treatment of diabetes. Partly because multiple applications may be developed and managed by different entities, healthcare action items generated by these healthcare applications may conflict with each other. Alternatively or in addition, the healthcare action items may be redundant, inefficient or become too complicated to be performed by patients or visitors of the medical station 140.

Network 110 is a set of devices that enable computing devices to communicate with each other. Various communication protocols may be used by network 110 including, but not limited to, Internet Protocols, Ethernet and wireless network protocols such as cellular standards.

The architecture of FIG. 1 is merely illustrative and various changes may be made. For example, backend server 120 may be combined with application developer devices 130. Also, some medical stations 140 may communicate with backend server 120 via another medical station 140.

FIG. 2 is a block diagram illustrating components of backend server 120, according to one embodiment. Backend server 120 may include, among other components, processor 202, memory 210, network interface 206 and bus 250 connecting these components. Backend server 120 may include other components not illustrated in FIG. 2.

Processor 202 reads and executes instructions stored in memory 210 to perform various operations. Although only a single processor 202 is illustrated in FIG. 2, multiple processors may be included in backend server 120.

Network interface 206 is hardware or hardware in combination with firmware or software for communicating with medical stations 140 and/or application developer devices 130. For this purpose, network interface 206 may implement various wired or wireless protocols.

Memory 210 is a non-transitory storage medium for storing software modules. Memory 210 may include, among other software modules, application repository 212, patient information module 220, medical station management module 230, treatment assessment module 240, action information reception module 244 and action management module 248. Memory 210 may store other software modules not illustrated in FIG. 2. Two or more software modules in FIG. 2 may be combined into a single module or a software module in FIG. 2 may be split up into multiple software modules.

Application repository 212 stores applications 280 to be sent to medical stations 140 for installation. Application repository 212 may retain the most recent versions of the applications 280 and may archive older versions of the applications 280. Newer applications or updated versions of the applications 280 may be automatically received from application developer devices 130 and be stored in application repository 212. In addition or alternatively, applications 280 may be transferred and stored in application repository 212 manually by human operators. Application repository 212 may also store metadata on applications 280, including but not limited to, related applications, stored versions of applications, ID of developers who developed the applications, and eligible membership to access the applications.

In other embodiments, backend server 120 does not include application repository 212. Rather, medical stations 140 receive applications directly from application developer devices 130 via network or through manual installation.

Patient information module 220 is a database including the list of patients and their profile information (e.g., name, age, gender,, insurance information). Patient information module 220 may also include EMR of the patients that may be updated based on diagnostic operations performed at medical stations 140. Likewise, EMR or other patient information can be updated via a mobile device, via traditional medical facility visits, via a web interface, via a health app, or via any other suitable ways. Patient information module 220 may be accessed by medical stations 140 to determine whether a patient should be granted access to medical stations 140 and/or certain applications 280 executable on medical stations 140. Further, patient information module 220 may store information on healthcare action items received from action information reception module 244, applications 280 and/or action management module 248.

Medical station management module 230 is a software module that performs various management operations related to medical stations 140. Medical station management module 230 may keep track of inventories of medical tools available in medical stations 140 and take actions to replenish them if the inventories are running low. For this purpose, medical station management module 230 may send orders to warehouses to ship tools 348 to sites where medical stations 140 are located. Also, medical station management module 230 may instruct maintenance personnel to visit and carry out predetermined maintenance operations on a certain medical station.

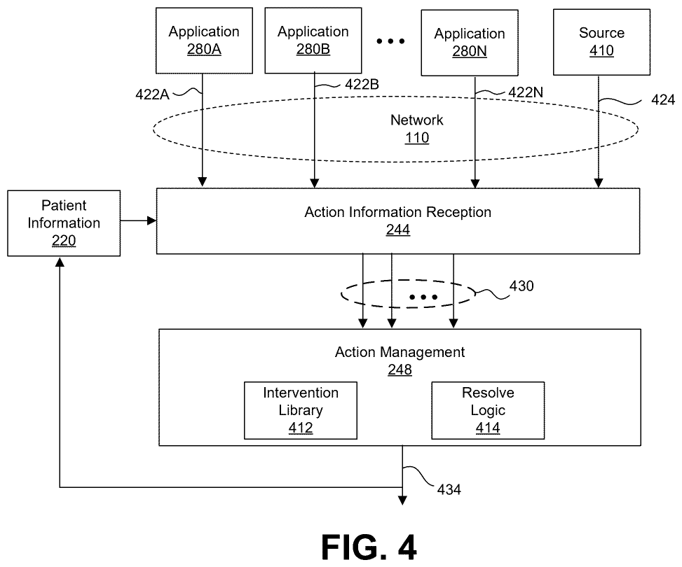

Treatment assessment module 240 performs analysis to determine the efficacy of treatments performed on patients. For this purpose, treatment assessment module 240 may receive patients' information from one or more medical stations 140. The treatments analyzed by treatment assessment module 240 may be part of healthcare action items generated by applications 280 executed on medical stations 140. Various statistical analyses may be performed on the patients' information to determine the efficacy or performance of treatment options. In some embodiments, the distribution of multiple medical stations 140 as described herein allows users to access care more frequently than having to visit traditional doctor's offices, and allows for a greater collection of patient data. Likewise, the medical stations 140 described herein enable more and different types of patient data to be collected. The medical stations 140 can leverage this data to evaluate adherence and effectiveness of particular treatment options, which in turn can be leveraged to evaluate best courses of action or treatment to recommend to other users. The information of the patients or visitors may be provided in real-time to the treatment assessment module 240 to enable health service providers to evaluate, assess and choose treatment options. In addition, the information of the patients or visitors (such as demographic or geographic information, health risks, existing conditions, history, and the like) may be leveraged to recommend certain health apps or programs offered by the medical stations 140. Treatment assessment module 240 may also provide information to action management module 248 to update any relationships between medical interventions, as described below in detail with reference to FIG. 4.

Action information reception module 244 is a software module that interfaces with medical stations 140 and/or other sources to receive healthcare action items. Healthcare action items are generated by applications 280 executed on medical stations 140 by processing diagnostic information and/or patient information. The diagnostic information may be obtained by using various tools 348 or plug-in components 370 available on medical stations 140. Healthcare action items may also be available from other sources such as medical professionals or healthcare facilities other than medical station 140. Alternatively or in addition, the diagnostic information may be extracted from patient information module 220 stored in backend server 120 or other healthcare facilities. Action information reception module 244 sends the healthcare action items relevant to a patient or a visitor to action management module 248.

Action management module 248 is a software module that manages the healthcare action items. Specifically, action management module 248 receives healthcare action items from different sources (e.g., applications 280, patient information module 220 and healthcare facilities) and converts the received healthcare action items into updated healthcare action items to reduce or eliminate complications associated with multiple different healthcare action items or generate an updated healthcare action items that combines multiple healthcare action items for increased efficacy and/or efficiency. Each of applications 280 or other sources may generate or provide different healthcare action items to address the health condition that they are associated with. The complications may be caused by conflicts between two or more of the healthcare action items, redundancy or overlapping of the healthcare action items, and increased complexity associated with performing two or more healthcare action items, as described below in detail with reference to FIG. 4.

FIG. 3 is a block diagram of medical station 140, according to one embodiment. Medical station 140 may include, among other components, computing device 320, one or more display devices 330, input device 332 for receiving user input, drawer assemblies 340, sensors 350, network interface 360 and plug-in components 370. Medical station 140 may include other components not illustrated in FIG. 3 such as a turntable on which a patient stands for scanning of the patient's body with a body scanner, interfaces that enable third-party or modular equipment or sensors to couple with and provide data/power to and from the equipment, and any other suitable equipment that extends the functionality of the medical station 140.

Computing device 320 executes logic to interface and control other components of medical station 140. For this purpose, computing device 320 may include, among other components, processor 322, component interface 324, and memory 326. Processor 322 is a hardware circuit or hardware circuit in combination with firmware that executes instructions stored in memory to perform various functions on computing device 320. Memory 326 is a non-transitory storage medium for storing software modules executable on processor 322. Component interface 324 is hardware or hardware in combination with software that enables interfacing with medical tools 348 (as applicable), sensors 350, and/or plug-in components 370. Component interface 324 may include multiple subunits that enable computing device 320 to communicate with different components of medical station 140 over different protocols.

Display device 330 receives display signals from computing device 320 to display various images to a patient or a human assistant. In one embodiment, one of display devices 330 may be a touch screen that is embedded onto a wall of medical station 140. Multiple display devices may also be included in medical station 140. For example, a display device may be installed at an entry of medical station 140 to display information related to access to medical station 140 and/or types of medical services provided by medical station 140 while another device installed in the interior of medical station 140 may display graphical user interfaces to select and receive a specific medical service.

Input device 332 is a device for receiving user inputs. Input device 332 may be embodied as part of a display device (e.g., a touch sensor) or a separate component (e.g., a microphone, a mouse or a keyboard). More than one input device may be included in medical station 140 to support different types of user inputs.

Drawer assemblies 340 are mechanically operated drawers that enable a patient or a human assistant to selectively access a medical tool or medical supplies. A drawer assembly may be locked or opened through operation of actuator 344 that operates according to a control signal from computing device 320. Medical tools 348 may include, among others, wireless heart rate monitor, digital stethoscope, contactless thermometer, pap smear, blood draw kit, vaccine supplies, ear lavage, dermatoscope, or any other suitable device or equipment for self-administration or administration by a healthcare provider. At least some of these medical tools 348 communicate via component interface 324 to enable collection of patient information in real-time while other medical tools 348 are collected for further testing and analysis. In some embodiments, the drawer assemblies 340 function as an API, allowing third-party entities to interface with the medical station 140 by, for example, specifying drawer requirements (such as a size, temperature control, power requirements, and the like) needed for providing medicine, treatments, or testing equipment to an individual.

Sensors 350 are provided in medical station 140 to detect physical characteristics or pose of the patients. In one embodiment, sensors 350 may be embodied as a body scanner that detects dimensions of various parts of the patient's body. The body scanner may be used in conjunction with a turntable (not shown) that rotates the patient while the scanning is being performed. A glucose meter may also be provided in medical station 140 as one of sensors 350 to detect the patient's blood level with or without drawing blood from the patient. Additional sensors 350 can include a thermography sensor, a blood pressure measurement system, a blood oxygenation detection system, a heart rate monitor, a body position sensor, a glucose meter, or any other suitable sensor or measurement/detection system.

Network interface 360 is hardware or hardware in combination with firmware that enables medical station 140 to communicate with backend server 120 or application developer devices 130 via network 110. Network interface 360 may, for example, include antenna and circuits for communicating over the Internet via wireless communication and/or a network card for communicating via wired communication (e.g., Ethernet) or for communication via private networks or peer-to-peer.

Plug-in components 370 are physical components added to medical station 140 to expand functionality to medical station 140. Plug-in components 370 may include a digital stethoscope, a digital dermatoscope, a pap smear kit, an EKG machine, an ultrasound device, a spirometer, a retinoscope, a blood draw kit, treatment kits (such as a cryo-gun, an ear lavage kit, a surgery kit, and the like), exercise materials (such as resistance bands), food and drinks, merchandise, prescription medicine, over-the-counter medicine, educational or informative materials, and the like. Functionalities of the medical station 140 that may leverage plug-in components 370 include but are not limited to: generating a 3D body model, measuring blood oxygenation, measuring/listening to heart sounds, performing venipuncture, measuring weight or height, administering vaccines or other injections, performing a nasal swab, collecting a urine sample, measuring blood pressure, observing insides of a patient's ears/nose/mouth/throat, capturing images of a patient's skin, performing an ultrasound/x-ray/MRI on a patient, observing a patient's eyes (e.g., using an ophthalmoscope), performing an EKG, performing a CT scan, performing spirometry functions, analyzing a patient's posture, performing cardiac telemetry, performing tonometry functions, capturing retinal or other images of the patient's eyes or other organs/body parts, capturing thermal or hyperspectral images of the patient, and analyzing a patient for chemicals or other compounds.

Memory 326 stores applications 280 that are software modules for interacting with the patients or visitors to provide medical or health related services to the patients or visitors. Each application 280 may be focused on different aspects of the medical/health services (e.g., a mental health checkup service, and a cardiovascular health checkup service). In some embodiments, a different suite of applications developed and managed by different health care entities may be stored in memory 326. At least some of these applications 280 are developed and received from application developer devices 130 via backend server 120.

Some of the applications 280 may make one or more tools 348 in drawer assemblies 340 available to the patients during their operations. For this purpose, the applications 280 may cause component interface 324 of computing device 320 to timely send activation signals to appropriate drawer assemblies 340 while the applications 280 are active. If multiple medical tools 348 are to be accessed by a patient, the active application may send a series of activation signals to activate actuators 344 of relevant drawer assemblies 340 so that relevant medical tools 348 may be sequentially accessed by the patient.

Applications 280 may also generate healthcare action items associated with the patient or visitor of medical station 140. Some of the healthcare action items may be in the form of operations performed on the patient or the visitor by plug-in components 370. The use or access to certain plug-in components 370 may be restricted to a subset of applications. For example, a plug-in component specifically designed for a medical service provider may be accessed or used only by an application developed or affiliated with the same medical service provider. Other healthcare action items may be in the form of follow-up actions to be taken by the patient, visitor or other entities during or after the patient or visitor accesses medical station 140.

Memory 326 also stores application management module 374. Application management module 374, when executed by processor 322, monitors healthcare action items generated by applications 280. When a healthcare action item is generated by an application, application management module 374 associates related information (e.g., patient ID) and sends the generated healthcare action item to action information reception module 244 of backend server 120 for processing. In one or more embodiments, application management module 374 may perform some or entire functions of action management module 248, described below with reference to FIG. 4.

FIG. 4 is a conceptual diagram of managing healthcare action items, according to one embodiment. Action information reception module 244 of backend server 120 receives healthcare action items 422A through 422N, and 424 from applications 280A through 280N and another source 410. Applications 280A through 280N may be deployed and executed in medical station 140. Source 410 may be medical professionals, medical service providers, users (e.g., patients) of medical station 140, and various government or public entities that provide their own healthcare action items 424. The users of medical station 140 or other sources 410 may provide information using, for example, applications executed on mobile or stationary computing devices. Although only a single source 410 is illustrated in FIG. 4, many other sources may provide their healthcare action items to action information reception module 244.

Action information reception module 244 receives healthcare action items, and forwards the healthcare action items to action management module 248. In one or more embodiments, action information reception module 244 references patient information module 220 to filter the received healthcare action items and forward only relevant healthcare action items 430 to action management module 248. For example, action information reception module 244 may remove healthcare action items that are outdated or obsolete (e.g., by taking a surgery or other medical interventions), and then sends only the healthcare action items that remain pertinent to the patient or visitor, as indicated by patient information module 220.

Action management module 248 converts received healthcare action items 430 into one or more updated healthcare action items 434 with complications associated with multiple healthcare action items removed or reduced relative to received healthcare action items 430 or combines multiple healthcare action items into fewer healthcare action items for increased efficacy or efficiency. The complications of multiple healthcare action items may be caused by conflicts between two or more of the healthcare action items, redundancy or overlapping of the healthcare action items, or increased complexity associated with performing two or more healthcare action items. By removing or reducing the complications, the efficiency or effectiveness of healthcare action items may be increased.

The conflicts between healthcare action items refer to inconsistency or contradiction in the healthcare action items. In some cases, the conflicts are apparent without further information. For example, a healthcare action item generated by one application for a person may recommend taking a certain medication while another healthcare action item generated by another application for the same person may recommend not taking the same medication. In other cases, the conflicts may not be so apparent. For example, two different applications may recommend taking two distinct medications that have adverse medical interactions. In these cases, obtaining additional information on the adverse medication interactions may be required to determine or identify the conflicts.

Redundancy or overlapping of the healthcare action items refers to a case where performing one healthcare action item renders another healthcare action item unnecessary or redundant. As in the case of conflicts, the redundancy or overlap may be apparent without further information. For example, a healthcare action item may recommend doing a certain exercise 5 minutes a day while another healthcare action item recommends doing the same exercise 30 minutes a day. In such case, it is apparent that the former is redundant and can be covered by the latter exercise of 30 minutes. In other cases, the redundancy or overlap may not be apparent without further information. For example, a healthcare action item may prescribe one type of anti-inflammatory medication while another healthcare action item may prescribe another type of anti-inflammatory medication that has the same primary effect as the former. In such case, the fact that the two prescriptions are duplicative may not be apparent without further information that the two medications belong to the same category of medications.

The complexity of performing the healthcare action items may increase when they are to be performed in conjunction. Taking the example of two different healthcare action items, each involving taking of a number of pills in a certain sequence after a meal, the number of medications may make it difficult to remember and take all the pills in a correct sequence. As in this case, the increase in the complexity of performing the healthcare actions may be more apparent when they are associated with the same type of actions (e.g., taking medications or performing physical exercises).

Action management module 248 identifies such complications associated with performing multiple healthcare action items, and converts these healthcare action items into updated healthcare action items to reduce or eliminate the complications. In one or more embodiments, action management module 248 determines priorities of different actions by predicting impact of each of the actions on the health of the patient or visitor, and chooses an action of a higher priority over other actions with lower priorities. Action management module 248 may also detect overlapping actions in the initial healthcare action items and select a superset of the overlapping actions as the updated healthcare action. Action management module 248 may also alert medical professionals of any conflicts or complications that action management module 248 is incapable of resolving, so that the medical professionals may review any conflicts or complications and resolve them. In one or more embodiments, action management module 248 addresses the conflicts of the healthcare action items by prioritizing the healthcare action items and eliminating the ones with lower priority. Action management module 248 may also merge the healthcare action items to address redundancy or overlapping. For example, in the case of recommending the same exercise with different time limits, action management module 248 may merge the healthcare action items with a shorter time into the healthcare action items with a longer time. Also, action management module 248 may decrease the complexity of performing the healthcare action items by prioritizing, eliminating and/or merging the healthcare action items. For example, in the case of healthcare action items that involve taking multiple sets of pills in a certain sequence, action management module 248 may identify a single pill that includes ingredients of these pills, and convert the two healthcare action items to an updated healthcare action item that prescribes a single pill with the multiple ingredients.

To convert the multiple healthcare action items, action management module 248 may include, among other components, intervention library module 412 and resolve logic module 414. Intervention library module 412 is a database that stores interactions and/or interchangeability between various medical interventions, priority of healthcare action items and other information associated with healthcare action items. Intervention library module 412 may update its stored information based on updated healthcare protocol and/or analysis performed by treatment assessment module 240. In one or more embodiments, intervention library module 412 may store, among others, information on adverse medication interactions, types and hierarchy of medical interventions, and priority or relationship between medical interventions, and statistical data on interactions between healthcare action items and their efficacy derived from, for example, healthcare applications or medical stations. Intervention library module 412 provides additional information to logic module 414 for detecting the complications that are not apparent as well as supporting intervention library module 412 to convert the received healthcare action items 430.

Resolve logic module 414 performs a set of logical operations to generate one or more updated healthcare action items 434 based on received healthcare action items 430. For this purpose, resolve logic module 414 accesses intervention library module 412 and performs logical operations on received healthcare action items 430. In one or more embodiments, resolve logic module 414 may be embodied using a rule-based system. After multiple healthcare action items are fed to the rule-based system, it detects complications associated with multiple healthcare action items and applies predetermined rules to remove some of the conflicting healthcare action items, modify the sequence of the healthcare action items or replace one or more of the healthcare action items.

Updated healthcare action items 434 generated by action management module 248 may be provided to the patient or visitor. Further, updated healthcare action items 434 may be provided to patient information module 220 to update its information. The updated patient information may be later used for filtering healthcare action items in a subsequent cycle. Alternatively or in addition, updated healthcare action items 434 may be provided to other entities (e.g., pharmacy or medical professionals) so that tasks associated with updated healthcare action items 434 may be taken.

By addressing the complications associated with multiple healthcare action items 430, embodiments may increase the efficacy of the healthcare action items and improve the compliance of the patient or visitor while reducing adverse effects or complexity associated with performing applicable healthcare action items. Further, treatment or diagnosis of health issues may be accomplished in a more effective manner.

In one or more embodiments, one or more updated healthcare action items 434 may be subject to verification to confirm that they do not include new complications or issues. For this purpose, multiple updated healthcare action items 434 may be again fed back to action management module 248 to further improve the healthcare action items 434. In addition or alternatively, a manual human review may be performed on the updated healthcare action items 434 to identify and/or resolve issues in the updated healthcare action items.

Although operations associated with generating the updated healthcare action items 434 are described as being performed solely at backend server 120, in other embodiments, medical station 140 may perform all or some of the operations of action management module 248. Further, action management module 248 may be provided on other computing devices of medical facilities or other healthcare services.

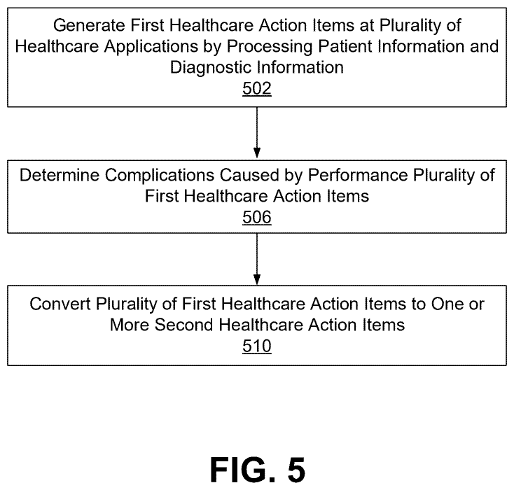

FIG. 5 is a flowchart illustrating operations of managing healthcare action items, according to one embodiment. A plurality of first healthcare action items are generated 502 for a patient or a visitor by processing patient information and diagnostic information of the patient or the visitor. The diagnostic information may be provided by a medical station that operates the healthcare applications and/or other sources such as medical professionals or other medical facilities.

Complications caused by performance of the plurality of first healthcare action items on the patient or the visitor are determined 506. The determination may be made by action management module 248 of backend server 120, taking into account patient information and interactions or interrelationships between medical interventions. The complications may include, among others, conflicts between the plurality of first healthcare action items, redundancy in the plurality of first healthcare action items, and increase in complexity associated with performing the plurality of first healthcare action items.

The first healthcare action items may be converted 510 into one or more second healthcare action items where the performance of the one or more second healthcare action items causes reduced complications relative to the performance of the plurality of first healthcare action items. Specifically, the conversion may be performed by removing a subset of the plurality of first healthcare action items according to priority, merging at least a subset of the plurality of first healthcare action items, modifying a sequence of performing the plurality of the first healthcare action items, and replacing one or more of the first healthcare action items.

The operations of FIG. 5 are merely illustrative. Additional processes may be added or some processes of FIG. 5 may be omitted. For example, a process of verifying the converted second healthcare action items may be added. Further, the sequence of processes in FIG. 5 may be modified. For example, the process of determining 506 complications and converting 510 the first healthcare action items may be performed in a single process or be performed in parallel.

While particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure.

What is claimed is:

1. A method of managing healthcare action items, comprising:
generating a plurality of first healthcare action items for a patient or a visitor of a medical station by at least a plurality of healthcare applications using patient information and diagnostic information of the patient or the visitor;
determining complications caused by performance of the plurality of first healthcare action items on the patient or the visitor; and
converting the plurality of the first healthcare action items to one or more second healthcare action items responsive to determining the complications, performance of the one or more second healthcare action items reducing or eliminating the complications relative to performance of the plurality of first healthcare action items.

2. The method of claim 1, wherein each of the first healthcare applications covers different fields of healthcare operations performed by the medical station.

3. The method of claim 1, wherein at least a subset of the first healthcare action items is received from one or more of medical professionals, medical service providers, medical publications or public entities.

4. The method of claim 1, wherein the complications caused by the performance of the plurality of first healthcare action items comprises at least one of: conflicts between the plurality of first healthcare action items, redundancy in the plurality of first healthcare action items, and increase in complexity associated with performing the plurality of first healthcare action items.

5. The method of claim 1, wherein the converting of the plurality of the first healthcare action items comprises at least one of:
removing a subset of the plurality of first healthcare action items according to priority, merging at least a subset of the plurality of first healthcare action items,
modifying a sequence of performing the plurality of the first healthcare action items, or
replacing one or more of the plurality of first healthcare action items.

6. The method of claim 1, further comprising storing, in a database, interactions or interchangeability between healthcare action items, wherein the converting of the plurality of the first healthcare action items is performed based on the stored interactions or the interchangeability.

7. The method of claim 6, wherein the converting of the plurality of the first healthcare action items comprises applying rules to the determined complications to generate the one or more second healthcare action items.

8. The method of claim 1, further comprising updating the patient information according to the one or more second healthcare action items.

9. The method of claim 1, further comprising:
performing management operations of the medical station, and
performing analysis to determine efficacy of treatments according to the first healthcare action items.

10. A computing device for managing healthcare action items, comprising:
one or more processors; and
a memory storing instructions thereon, the instructions when executed by the one or more processors cause the one or more processors to:
generate a plurality of first healthcare action items for a patient or a visitor of a medical station by at least a plurality of healthcare applications using patient information and diagnostic information of the patient or the visitor;
determine complications caused by performance of the plurality of first healthcare action items on the patient or the visitor; and
convert the plurality of the first healthcare action items to one or more second healthcare action items responsive to determining the complications, performance of the one or more second healthcare action items reducing or eliminating the complications relative to performance of the plurality of first healthcare action items.

11. The computing device of claim 10, wherein the computing device further comprises a network interface configured to receive the plurality of healthcare action items from the medical station via a network.

12. The computing device of claim 10, wherein each of the first healthcare applications covers different fields of healthcare operations performed by the medical station.

13. The computing device of claim 10, wherein at least a subset of the first healthcare action items is received from one or more of medical professionals, medical service providers, medical publications or public entities.

14. The computing device of claim 10, wherein the complications caused by the performance of the plurality of first healthcare action items comprise at least one of: conflicts between the plurality of first healthcare action items, redundancy in the plurality of first healthcare action items, and increase in complexity associated with performing the plurality of first healthcare action items.

15. The computing device of claim 10, wherein the plurality of the first healthcare action items are converted to the one or more second healthcare action items by performing at least one of:
removing a subset of the plurality of first healthcare action items according to priority, merging at least a subset of the plurality of first healthcare action items,
modifying a sequence of performing the plurality of the first healthcare action items, or
replacing one or more of the plurality of first healthcare action items.

16. The computing device of claim 10, further comprising a database configured to store interactions or interchangeability between healthcare action items, wherein the one or more processors convert the plurality of the first healthcare action items to the one or more second healthcare action items based on the stored interactions or the interchangeability.

17. The computing device of claim 16, wherein the instructions further cause the one or more processors to convert the plurality of the first healthcare action items by applying rules to the determined complications to generate the one or more second healthcare action items.

18. The computing device of claim 10, wherein the instructions further cause the one or more processors to update the patient information according to the one or more second healthcare action items.

19. The computing device of claim 10, wherein the instructions further cause the one or more processors to:
perform management operations of the medical station, and
perform analysis to determine efficacy of treatments according to the first healthcare action items.

20. A non-transitory computer-readable storage medium storing instructions thereon, the instructions when executed by one or more processors cause the one or more processors to
generate a plurality of first healthcare action items for a patient or a visitor of a medical station by at least a plurality of healthcare applications using patient information and diagnostic information of the patient or the visitor;
determine complications caused by performance of the plurality of first healthcare action items on the patient or the visitor; and
convert the plurality of the first healthcare action items to one or more second healthcare action items responsive to determining the complications, performance of the one or more second healthcare action items reduce or eliminate the complications relative to the performance of the plurality of first healthcare action items.
